(19) United States
(12) Patent Application Publication
(10) Pub. No.: US 2022/0074850 A1
(43) Pub. Date: Mar. 10, 2022

(54) GAS DETECTION SYSTEM

(71) Applicant: CARRIER CORPORATION, Palm Beach Gardens, FL (US)

(72) Inventors: Dayne Plemmons, Minneapolis, MN (US); Anand Venkatesh Sankarraj, Eden Prairie, MN (US); Nathan Herman, Chanhassen, MN (US)

(21) Appl. No.: 17/116,130

(22) Filed: Dec. 9, 2020

Related U.S. Application Data
(60) Provisional application No. 63/074,686, filed on Sep. 4, 2020.

(51) Int. Cl. G01N 21/3504; G01N 33/00

(52) U.S. Cl. CPC G01N 21/3504; G01N 2201/0631; G01N 2201/0636; G01N 33/0027

(57) ABSTRACT

Methods and systems for gas detection are provided. Aspects include determining a target gas for detection in a sampling chamber, determining one or more target characteristics of light based on the target gas, operating a light source to transmit the light through a sampling chamber to an optical element, operating an active optical element to modulate the light based on the one or more target characteristics of the light, operating a filter to receive the light from the optical element and separate the light in to a first light portion and a second light portion, operating the photodetector to receive the first light portion from the filter, and analyzing the first light portion to determine a presence of the target gas in the sampling chamber.

### CROSS-REFERENCE TO RELATED APPLICATIONS

This application claims the benefit of U.S. Patent Application No. 63/074,686 filed Sep. 4, 2020, which is incorporated herein by reference in its entirety.

### BACKGROUND

Exemplary embodiments pertain to the art of gas detection systems and more specifically to a gas detection system employing semi-broadband light and a wavelength modulated band-pass filter.

Gas sensors have been used in various applications such as process monitoring and control and safety monitoring. As certain gases can be flammable, toxic, or explosive, gas detection sensors have also been used for leak detection where such gases are used or manufactured. Various types of sensors and systems have been used or proposed, including but not limited to metal oxide semiconductor (MOS) sensors, non-dispersive infrared detector (NDIR) sensors, pellistor (pelletized resistor) sensors, oxygen ion-permeable high-temperature solid electrolytes, and electrochemical cells. Gas detection systems often utilize sensitive compositional information encoded into infrared absorption signatures. That is to say, light (often infrared light) is passed through a medium suspected of containing a certain type of gas and received by a photodetector. The photodetector provides data associated with the changes to the original light. This data is compared to absorption properties of different gases to determine the presence of a gas and the type of gas as well as other information such as, for example, the concentration of the gas. With these gas detection systems, there is often a compromise between selectivity, sensitivity, and cost.

### BRIEF DESCRIPTION

Disclosed is a method for gas detection. The method includes determining a target gas for detection in the sampling chamber, determining one or more target characteristics of the light based on the target gas, operating a light source to transmit light through a sampling chamber to an objective optical element, operating an optical filter to modulate the light based on the one or more target characteristics of the light, operating a filter to receive the light from the objective optical element and separate the light into a first light portion and a second light portion, operating the photodetector to receive the first light portion from the filter, and analyzing the first light portion to determine a presence of the target gas in the sampling chamber.

Disclosed is a system for gas detection. The system includes a light source operable to transmit light through a sampling chamber to an optical element, an active optical element operable to modulate one or more characteristics of the light, a filter operable to receive the light from the objective optical element, a photodetector, and a controller configured to determine a target gas for detection in the sampling chamber, determine one or more target characteristics of the light based on the target gas, and operate the optical filter to modulate the light based on the one or more target characteristics of the light.

### BRIEF DESCRIPTION OF THE DRAWINGS

The following descriptions should not be considered limiting in any way. With reference to the accompanying drawings, like elements are numbered alike:

FIG. 1 depicts a block diagram of a system for gas detection according to one or more embodiments;

FIG. 2 depicts an exemplary absorption graph for gases according to one or more embodiments; and

FIGS. 3a and 3b depict graphical representations of wavelength modulation according to one or more embodiments;

FIG. 4 depicts a flow diagram of a method for gas detection according to one or more embodiments;

FIG. 5 depicts a flow diagram of a method for gas detection according to one or more embodiments; and

FIGS. 6a and 6b depict an illustrative schematic of a method for gas detection according to one or more embodiments.

The diagrams depicted herein are illustrative. There can be many variations to the diagrams or the operations described therein without departing from the spirit of the disclosure. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

### DETAILED DESCRIPTION

A detailed description of one or more embodiments of the disclosed apparatus and method are presented herein by way of exemplification and not limitation with reference to the Figures.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

Turning now to an overview of the aspects of the disclosure, one or more embodiments provide for a gas detection system with an electrically-tunable band-pass filter which selectively transmits light from a semi-broadband light-source (e.g., light emitting diode (LED)) to a photodetector through a sampling chamber. The spectral position of the peak light emission from the light source and transmission of the filter can be chosen to coincide with an absorbing region of the infrared spectrum of a gas that may or may not be within the sampling chamber. By modulating the filter's transmission wavelength characteristics across a gas-absorption region while synchronously modulating the amplitude of the light (either by current control or as dictated by the spectral properties of the infrared source), the gas detection system can extract sensitive gas concentration measurements of a gas present in the sampling chamber from the amplitude and phase-dependence of the photodetector measurements (i.e., the frequency domain characteristics of the resultant waveform signal). The selectivity for and sensitivity to numerous gasses can be achieved by tuning characteristics of the filter and the light source, i.e., adjusting the wavelength modulation bandwidth, the wavelength peak center, the phase relation, and the source spectrum, for example.

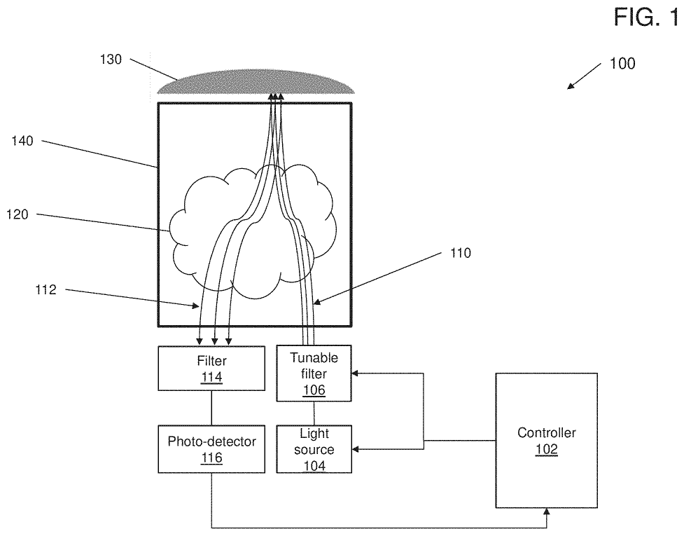

Turning now to a more detailed description of aspects of the present disclosure, FIG. 1 depicts a block diagram of a system for gas detection according to one or more embodiments. The system 100 includes a controller 102. The controller 102 is configured to operate and communicate with a light source 104, an active optical element, such as, a tunable filter 106, and a photodetector 116. The system 100 also includes a sampling chamber 140, an optical element 130, and a filter 114. As mentioned above and in one or more embodiments, the system 100 is configured to determine the presence and/or characteristics of a target gas 120 in the sampling chamber 140. In one or more embodiments, the system 100 is configured to detect a target gas. For example, in an industrial setting, it may be desirable to configure the system 100 to detect the presence of methane gas due to the potential for methane to combust. In this setting, the system 100 and the various components would be configured to detect methane and, in reference to FIG. 1, the target gas 120 would be methane. The system 100 would then be able to detect the methane (i.e., target gas 120) when/if methane were to enter the sampling chamber 140. Further, in reference to this example, since the target gas 120 is known when setting up the system 100, other properties and characteristics can be determined for the methane such as concentration and the like.

Referring back to FIG. 1, the controller 102 is configured to obtain data from the photodetector 116 and filter 114. This data includes one or more characteristics of light from the light source 104 when passed through the sampling chamber (and through the target gas 120, if present). By analyzing this data, the controller 102 is operable to determine one or more characteristics of the target gas 120 when present in the sampling chamber 140. When the target gas 120 is not present within the sampling chamber 140, the system 100 will not detect any other gases that may be present within the sampling chamber. The controller 102, in one or more embodiments, may determine the presence of the target gas 120 and other information such as, for example, volumetric concentration, partial pressure, parts per million of the target gas 120. In one or more embodiments, the optical element 130 may be a mirror which reflects light back through the gas sampling chamber 140. The mirror may be spherical or parabolic so as to focus the light towards the photodetector 116.

In one or more embodiments, the controller 102 is configured to operate the light source 104 to vary the intensity (amplitude) of the light being emitted. In some embodiments, the light source can be any type of light source such as, for example, an LED, micro-hot-plate, infrared light source, and the like. The light source 104 can be coarsely tunable via temperature control or current control of the light source. The light source 104 emits light over a broad range of wavelengths. The tunable filter 106 is operated by the controller 102 to modulate the transmission wavelength characteristics of the light across a spectrum. That is to say, the controller 102 determines a range of wavelengths for the tunable filter 106 to filter the light from the light source 104. The range of wavelengths is selected based on the target gas 120 absorption characteristics, which are known in the art. When the range of wavelengths is determined, the light passing through the tunable filter 106 is restricted to the wavelengths within that range. For example, when detecting hydrogen sulfide (H2S), the wavelength for detection is known in the art as being a subset of wavelengths between 1550 nm to 1600 nm. The tunable filter 106, in this example, would restrict the light emitted into the sampling chamber to be within a narrow region of this range where H2S, the target gas, absorbs light. The subsets of wavelengths for detection of other gasses are known in the art and thus the tunable filter 106 may be adjusted to detect other target gasses as described herein.

In one or more embodiments, the tunable filter 106 can be any type of electrically tunable filter such as, for example, a bandpass filter, a MEMS Fabry-Perot interferometric filter, a metal-dielectric meta-surface, and the like. In one or more embodiments, the light 110 emitted from the light source 104 and modulated by the tunable filter 106 is transmitted through the sampling chamber 140 and through the target gas 120 (if present) and reflected off the objective optical element 130. The reflected light 112 is then passed back through the sampling chamber 140 and the target gas 120 to the filter 114. In some embodiments, the filter 114 can be an optical filter such as, for example, a notch filter or an out of band filter. The filter 114 is configured to collect the reflected light 112 into the photodiode (a first portion of the light) and filter out any outside light (second portion) that may affect the photodetector 116. That is to say, the filter 114 allows reflected light 112 to pass through if it is within a certain set of wavelengths (e.g., the first portion of the reflected light) and any light outside the set of wavelengths is filtered out (e.g., the second portion of the reflected light). The set of wavelengths is determined based on the target gas 120 selected for the system 100 to detect. The photodetector 116 is utilized to detect the first portion of the reflected light 112. The photodetector 116 is operated by and in communication with the controller 102. The light data collected by the photodetector 116 can be transmitted to the controller 102 for processing and analysis.

In one or more embodiments, the controller 102 may make adjustments to any of the various components of the system 100 to enable detection of a target gas 120. The type of gas set as the target gas 120 can be adjusted based on the gas detection application for the system 100. For example, gas detection operation may be for the detection of benzene. The controller 102 can receive known properties of benzene to determine a set of wavelengths and operational needs for the various components of the system 100 to detect benzene (as the now target gas 120) for detection within the sampling chamber 140. The sampling chamber 140 is typically placed in an area where there is a high probability for the presence of the target gas 120. The controller 102 can adjust the operation of any of the components of the system 100 to determine the presence of the target gas 120 (benzene, in the example) within the sampling chamber 140 and also the concentration and any other characteristics of the target gas (as determined from the data received from the photodetector 116 as explained below). Similarly, if a new target gas, methane for example, is selected for detection, the controller 102 can adjust the components of the system 100 to now detect the new target gas (e.g., methane) based on known properties of the new target gas. In addition, the operation of the various components in the system 100 can be based on the type of gas being determined to be the target gas 120. For certain types of gases (e.g., flammable, toxic, etc.), the sensitivity of the components can be adjusted by the controller 102 for the detection of these dangerous, target gases.

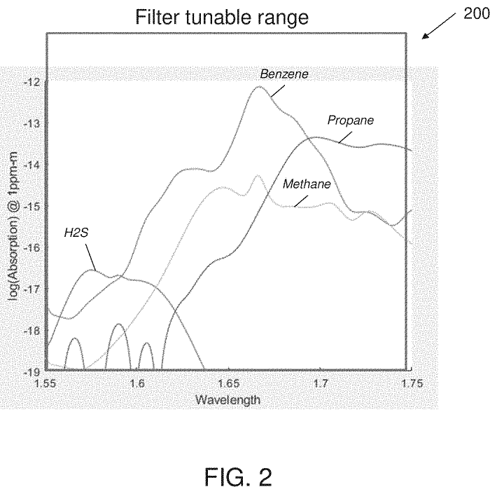

FIG. 2 depicts an exemplary absorption graph for gases according to one or more embodiments. Absorption graphs for gas detection are known in the art. The illustrated graph 200 includes a range of wavelengths from 1550 nm to 1750 nm and the corresponding absorption values for four exemplary gases. The example gases include hydrogen sulfide (H2S), benzene, propane, and methane. The absorption values are depicted on a log-scale for a 1 part per million concentration at 1 meter path-length; the indicated quantities are smoothed (averaged) with a 12-nm bandwidth Lorentzian low-pass filter indicative of an example tunable filter bandwidth. This graph 200 can be utilized to determine the range of wavelengths for detecting any of the four gases. For example, for H2S, as mentioned above, a range of wavelengths near 1560 nm can be selected to determine the presence of H2S within the sampling chamber 140 of the system 100. By adjusting the wavelength of the light emitted from the light source 104, the system 100 (from FIG. 1) can be utilized to detect H2S within the sampling chamber 140, when present. The tunable filter 106 can be adjusted to allow a light wavelength range from 1620 nm to 1710 nm to be emitted into the sampling chamber 140 to allow for the controller 102 to be able to detect the presence of any of the other three gases of benzene, propane, and methane when present in the sampling chamber 140. For sensitive and selective gas detection (e.g., H2S), the system 100 operates the tunable filter 106 to tune across a narrow portion of the wavelength spectrum where a gas of interest (i.e., target gas 120) is most sensitive relative to the other gases. In contrary, for generalized gas detection of a selected target gas, the system 100 can be operated such that the tunable filter 106 samples a relatively broad range of wavelengths where absorption occurs for a wide range of possible gases (e.g., for detection and quantification of flammable gas mixtures). This allows the system 100 to provide flexible gas detection that can be tuned for specific use-cases using firmware changes instead of wholesale hardware and design changes.

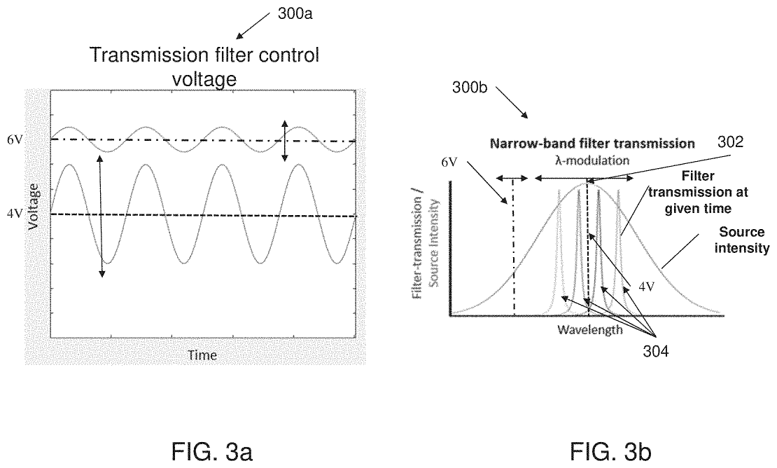

FIGS. 3a and 3b depict graphical representations of wavelength modulation according to one or more embodiments. The amplitudes of a transmission filter control voltage waveform are shown in FIG. 3a which includes graph 300a showing the amplitudes (arrows in FIG. 3a) with respect to the DC offsets for 4V and 6V. The modulation characteristics of the tunable filter 106, (such as the amplitude and central wavelength) with respect to the light source 104 emission spectrum and a target gas 120 absorption spectrum are operatively chosen for a given application based on sensitivity and selectivity criteria.

Graph 300b as shown in FIG. 3b depicts a narrow band filter transmission wavelength modulation example. The source intensity shows the light source 104 emission wavelength range. A DC-offset in the voltage control waveform dictates which transmission wavelength the tunable filter 106 will be modulated around. For example, the 4V DC offset will yield the wavelength range center at the peak of the source intensity 302 in the example schematic. The amplitude (arrows in FIG. 3b) of the 4V DC-offset control voltage waveform (dotted line) example dictates the range of central wavelengths across which the tunable filter 106 samples. As another example, a 6V DC-offset control voltage waveform (dashed line) results in central wavelength offset from the peak emission and with much narrower modulation amplitude (arrows in FIG. 3b). Note that each of the example narrow-band transmission lines 304 shown in graph 300b represent the tunable filter's transmission characteristics at a given point in time (for the 4V case, for instance).

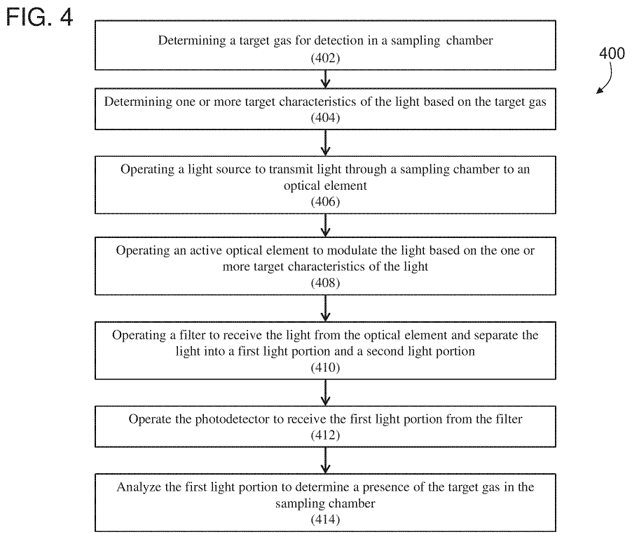

FIG. 4 depicts a flow diagram of a method for gas detection according to one or more embodiments. The method 400 includes determining, by the controller 102, a target gas for detection in the sampling chamber 140, as shown in block 402. The method 400, at block 404, includes determining one or more target characteristics of the light based on the target gas. The controller 102 can determine characteristics of light (e.g., wavelength, etc.) based on known data associated with the earlier determined target gas for detection by the gas detection system. Also, at block 406, the method 400 includes operating the light source 104 to transmit the light through a sampling chamber to an optical element. The method 400 also includes operating the tunable filter 106 to modulate the light based on the one or more target characteristics of the light, as shown at block 408. At block 410, the method 400 includes operating the filter 114 to receive the light from the optical element and separate the light into a first light portion and a second light portion. Also, the method 400, at block 412, includes operating the photodetector 116 to receive the first light portion from the filter 114. And at block 414, the method 400 includes analyzing the first light portion to determine a presence of the target gas in the sampling chamber.

Additional processes may also be included. It should be understood that the processes depicted in FIG. 4 represent illustrations and that other processes may be added or existing processes may be removed, modified, or rearranged without departing from the scope and spirit of the present disclosure.

In one or more embodiments, the photodetector 116 provides the controller 102 with the signal associated with detected light passing through the sampling chamber 140 and the filters 106 and 114. The controller 102 can analyze this data to determine the presence of a target gas as well as other characteristics. In one or more embodiments, the controller 102 can analyze the measured signal characteristics of the light passing through the target gas 120 at the photodetector 116 as compared to known values of those signal characteristics when the target gas is not present in the sampling chamber. In other embodiments, data collected at the photodetector 116 can be compared to known values of signal characteristics when a pre-calibrated quantity of the target gas is present. This pre-calibration can occur prior to the installing the gas detection system as a way of tuning for a particular target gas. Processing techniques (and extraction of exemplary signal characteristics) include, but are not limited to, comparing a Fourier transform of the light emitted from the light source 104 (after appropriate filtering and signal conditioning; e.g., DC-offset subtraction) to determine the power of the signal at specific frequencies. For example, ratios of specific harmonics of the modulation frequency (i.e., the frequency at which the tunable filter 106 cycles through its wavelength range) can be compared, by the controller 102, against a calibrated look-up table or graph to determine a characteristics of the target gas (e.g., volumetric concentration, partial pressure, parts per million).

Depending on the target gas selected for detection, the desired concentration measurement range, and the accuracy requirements for a given application, adjustments to the system 100 components (e.g., the transmission filter control voltage 300a dictating the properties of tunable filter 106, etc.) can be made to achieve suitable signal-to-noise (e.g., ratio of detection signal to background), sensitivity (e.g., the smallest quantity of target gas detectable) and signal-linearity (e.g., results directly proportional to target gas concentration) as required by the specific gas-detection application (e.g., combustible gas detection, etc.). In low sensitivity applications (in which light is strongly attenuated by the target gas by virtue of suitably high gas concentration), processing techniques may include comparing the amplitude of the modulated light at the modulation frequency with a known zero-gas amplitude (obtained during a calibration procedure). This ratio may be compared to known calibrated look-up values or graphs such that the gas concentration may be expressed in reference to known flammability limits (i.e., percent of lower-flammability-limit).

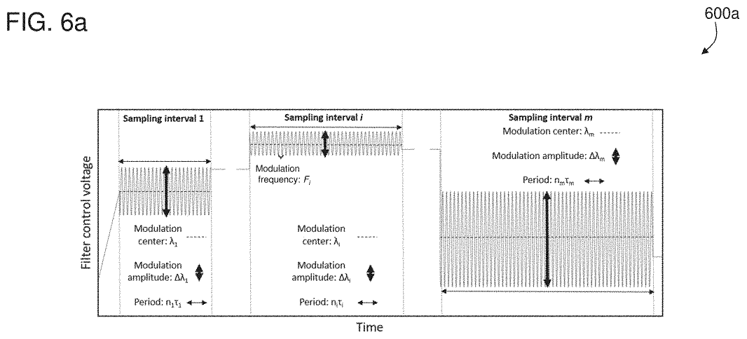

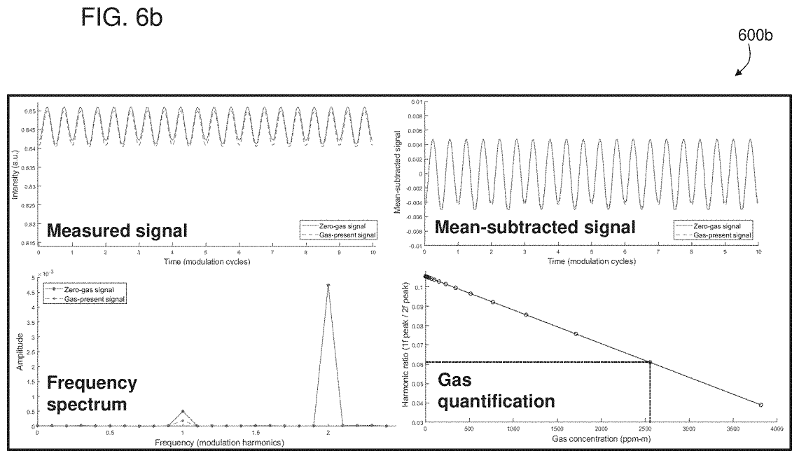

In some embodiments, gas detection and quantification will be realized via a system-specific variant of wavelength modulation spectroscopy. As shown in FIG. 6a, and described in further detail below, the wavelength of the tunable filter 106 (by way of the transmission filter control voltage 300a) is continuously modulated with a given frequency Fᵢ across a range of wavelengths Δλ centered at wavelength λ. The resultant waveform signal from the first portion of reflected light passed by filter 114 at the photodetector 116 is dependent on both the spectral properties of the light source 104 and the spectral features of absorbing gasses in the sampling chamber 140. If the light source 104 has a fixed optical spectrum and intensity, monitoring the subtle changes in the signal waveform from the photodetector 116 reveals sensitive information regarding presence and concentration of a target gas.

The controller 102 may analyze a waveform output by the photodetector 116. Analyzing the waveform from photodetector 116 in the frequency domain, the change in power concentrated at the first and second harmonic of the modulation frequency F is related to the slope and curvature, respectively, of the absorption spectrum of a target gas. In this manner, one may choose specific wavelength regions for tunable filter 106 modulation such that the gas absorption features impact the signal in robust fashion. At the inflection point near the onset of gas absorption, for instance, the slope of absorbance with respect to wavelength is large while the curvature is small; thus, the majority of signal change would occur in the first modulation frequency harmonic. Monitoring the power in the first harmonic (target-gas-dependent) relative to the second harmonic (largely target-gas-independent) serves as a route to gas quantification that is independent of the total signal—that is, gas concentration may be quantified in such a manner that fluctuations in the amount of light received at the photodetector 116 does not significantly impact the quantification procedure. In this manner, field-environment fluctuations in total light intensity which may result from obscurations in the light path (e.g., dirty optics, etc.) or variations in the light source 104 do not have a substantial impact on the accuracy of gas concentration measurements. In practice, appropriate modulation parameters or sets of modulation parameters for this system-specific variant of wavelength modulation spectroscopy are chosen such that the desired sensitivity and selectivity to a target gas or multiple or other target gasses is achieved.

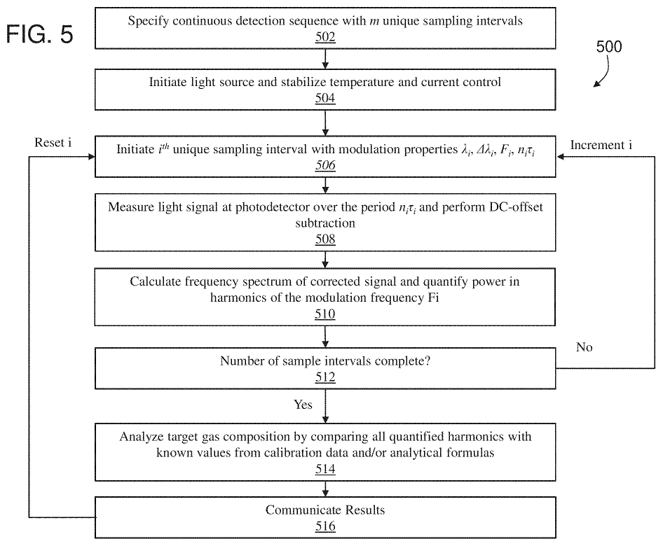

In one or more embodiments, the system 100 can be operated following the flow diagram outlined in FIG. 5 and illustrated schematically in FIG. 6a and FIG. 6b. In FIG. 5, a method 500 starts where controller 102 specifies a detection sequence consisting of m sampling intervals with unique modulation properties λᵢ, Δλᵢ, Fᵢ, nᵢτᵢ, as shown in block 502. These variables (properties of the transmission filter voltage control waveform 300a) represent the central filter wavelength (λᵢ), the modulation amplitude (Δλᵢ), the modulation frequency (Fᵢ), and the detection period (nᵢτᵢ). An example detection sequence 600a is shown in FIG. 6a with the variables illustrated schematically and labeled. These parameters and the required number of sampling intervals (m) may be chosen according to various application-specific cases outlined in following paragraphs. After specification of a given detection sequence, the method 500 includes that the corresponding light source 104 properties are fixed and stabilized such that the resulting time-domain signal is only dependent on the filter properties, as shown in block 504.

After setting the detection sequence (FIG. 6a) at 500 and allowing the light source 104 to stabilize at 504, the method 500, at block 506, continues where the continuous detection process is initiated in which the detector cycles through the m unique intervals and quantifies the gas properties after each completion of the sequence. The signal processing and analysis occurring during blocks 506 to 516 of FIG. 5 in a single sampling interval 600b as illustrated in FIG. 6b. The method 500 continues where the waveform from the light signal is recorded over a period nᵢτᵢ (FIG. 6b, upper-left panel) and the mean over that period (e.g., DC Offset) is subtracted (FIG. 6b, upper-right panel) from the signal such that the frequency spectrum (FIG. 6b, lower-left panel) may be calculated appropriately, as shown in block 508 of method 500. Amplitudes at particular harmonics of the modulation frequency are quantified and used in the analysis of the target gas properties as shown in block 510 of method 500. At block 512, the method 500 continues by determining whether the specified m sampling intervals (from block 502) have been completed. If the desired number of sample intervals have been completed (i.e., i=m), the method 500 continues to block 514. Method step 510 can be performed after the completion of each sampling interval i or can be performed for all intervals m after the detection sequence has been completed. Quantification of target gas or gas mixture concentrations consists of comparing some combination of measured harmonic amplitudes with calibration-determined quantities and extrapolating, such as the extrapolation example in the lower-right panel of FIG. 6b and included in block 514 of method 500. The lower-right panel shows the extrapolated relationship between an exemplary gas concentration and the harmonic ratio (e.g., first harmonic peak to the second harmonic peak). This relationship can be determined from look up tables and the extrapolation (e.g., the line in the lower-right panel) is done using known extrapolation techniques. After the detection sequence is complete and the gas concentration or concentrations have been quantified, the information is communicated to the user and the detection sequence is reinitiated, as shown in block 516 for method 500.

In one or more embodiments, a detection sequence 600a may include numerous sampling intervals with varying modulation parameters such that the pertinent spectral properties of the absorbing gas or gasses are examined in a manner suitable for a given detection application. For instance, one may choose to have a sampling interval corresponding to each of multiple gasses of interest. Each of these sampling intervals would be tuned according to the absorbing features of the multiple gasses of interest (e.g., aligning central wavelength λᵢ with a strongly absorbing feature unique to gas of interest i). Alternatively, one may choose to have multiple sampling intervals pertinent to a single gas but for the purpose of ruling out the presence of a known interfering gas. In this manner, additional confidence in the detection and gas quantification may be achieved without susceptibility to raising false alarms. Additionally, one may choose to have multiple sampling intervals pertinent to a single gas but for the purpose of taking measurements across a wide range of concentrations. One set of sampling intervals may be specific to a low-concentration range (i.e., parts-per-million level) where high sensitivity and selectivity is required (but signal-linearity is compromised at high concentrations), and another set of sampling intervals may be specific to higher concentrations in which signal-linearity is achieved over a larger portion of the measurement range (but may not have adequate sensitivity at the low-end as dictated by the system's signal-to-noise limits). One may also employ a composite metric from signal data acquired over the multiple sampling intervals during a detection sequence such that robust differentiation and quantification of unknown gasses or gas mixtures may take place. For this purpose, a detection sequence can be designed using statistical methods which best differentiate gasses of a given character (e.g. alkanes versus alkenes, aromatics versus aliphatics, etc.).

In one or more embodiments, the controller 102 or any of the hardware referenced in the gas detection system 100 can be implemented by executable instructions and/or circuitry such as a processing circuit and memory. The processing circuit can be embodied in any type of central processing unit (CPU), including a microprocessor, a digital signal processor (DSP), a microcontroller, an application specific integrated circuit (ASIC), a field programmable gate array (FPGA), or the like. Also, in embodiments, the memory may include random access memory (RAM), read only memory (ROM), or other electronic, optical, magnetic, or any other computer readable medium onto which is stored data and algorithms as executable instructions in a non-transitory form. Additionally, a network may be in wired or wireless electronic communication with one or all of the elements of the system 100. Cloud computing may supplement, support or replace some or all of the functionality of the elements of the system 100. Additionally, some or all of the functionality of the elements of system 100 may be implemented as a cloud computing node.

A detailed description of one or more embodiments of the disclosed apparatus are presented herein by way of exemplification and not limitation with reference to the Figures.

Various embodiments are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A system for gas detection, the system comprising:
a light source operable to transmit light through a sampling chamber to an optical element;
an active optical element operable to modulate one or more characteristics of the light;
a filter operable to receive the light from the optical element;
a photodetector; and
a controller configured to:
determine a target gas for detection;
determine one or more target characteristics of the light based on the target gas; and
operate the active optical element to modulate the light based on the one or more target characteristics of the light.

2. The system of claim 1, wherein operating the optical filter to modulate the light based on the one or more target characteristics of the light comprises:
modulating a wavelength of the light over across a range of wavelengths over a time period.

3. The system of claim 1, wherein the controller is further configured to:
operate the light source to transmit light through the sampling chamber to the objective optical element;
operate the filter to receive the light from the optical element and separate the light into a first light portion and a second light portion;
operate the photodetector to receive the first light portion from the filter;
analyze the first light portion to determine a presence of the target gas in the sampling chamber.

4. The system of claim 3, wherein operating the light source comprises:
modulating, by the controller, light intensity of the light across a spectrum.

5. The system of the claim 3, wherein the controller is further configured to:
based at least in part on determining the presence of the target gas in the sampling chamber, analyze the first light portion to determine one or more characteristics of the target gas.

6. The system of claim 5, wherein the one or more characteristics of the target gas comprises at least one of volumetric concentration, partial pressure and parts per million.

7. The system of claim 1, wherein the one or more target characteristics of the light comprises a range of wavelengths associated with the target gas.

8. The system of claim 1, wherein the active optical element comprises a tunable optical filter.

9. The system of claim 1, wherein the filter comprises a band-pass filter.

10. The system of claim 1, wherein the filter comprises a Fabry-Perot interferometric filter.

11. A method for gas detection, the method comprising:
determining a target gas for detection in a sampling chamber;
determining one or more target characteristics of light based on the target gas;
operating a light source to transmit the light through a sampling chamber to an optical element;
operating an active optical element to modulate the light based on the one or more target characteristics of the light;
operating a filter to receive the light from the optical element and separate the light in to a first light portion and a second light portion;
operating the photodetector to receive the first light portion from the filter; and
analyzing the first light portion to determine a presence of the target gas in the sampling chamber.

12. The method of claim 11, wherein operating the active optical element comprises:
modulating a light intensity of the light across a spectrum.

13. The method of the claim 11, further comprising:
based at least in part on determining the presence of the target gas in the sampling chamber, analyzing the first light portion to determine one or more characteristics of the target gas.

14. The method of claim 13, wherein the one or more characteristics of the target gas comprises at least one of volumetric concentration, partial pressure and parts per million.

15. The method of claim 11, wherein the one or more target characteristics of the light comprises a range of wavelengths associated with the target gas.

16. The method of claim 11, wherein the active optical element comprises an active optical filter.

17. The method of claim 11, wherein the filter comprises a band-pass filter.

18. The method of claim 11, wherein the filter comprises a Fabry-Perot interferometric filter.

19. The method of claim 11, wherein the optical element comprises a reflective element.

20. The method of claim 11, wherein the optical element comprises a mirror.
